(19) United States
(12) Patent Application Publication
(10) Pub. No.: US 2016/0115503 A1
(43) Pub. Date: Apr. 28, 2016

(54) PROCESS AND METHOD FOR IMPROVING FERMENTATION BY THE ADDITION OF HYDROTHERMALLY TREATED STILLAGE

(71) Applicant: VALICOR, INC., Dexter, MI (US)

(72) Inventors: James R. Bleyer, Maumee, OH (US); Thomas Czartoski, Tecumseh, MI (US)

(73) Assignee: Valicor, Inc., Dexter, MI (US)

(21) Appl. No.: 14/893,577

(22) PCT Filed: May 27, 2014
(86) PCT No.: PCT/US14/39537

§ 371 Date: Nov. 24, 2015

Related U.S. Application Data
(60) Provisional application No. 61/827,292, filed on May 24, 2013.

(51) Int. Cl. C12P 7/06; C12N 1/00

(52) U.S. Cl. CPC C12P 7/06; C12N 1/005; C12P 2201/00

(57) ABSTRACT

A method of improving fermentation by increasing the bioavailability of components in stillage, using all or a portion of the hydrothermally treated stillage as a component of a media, and using the media for a process such as fermentation or biomass production. The metabolites and biomass recovered from the method above. Media including hydrothermally treating stillage obtained by heating the stillage to a temperature of 190° F. to 300° F. A method of improving fermentation by removing solids in stillage, hydrothermally treating the stillage by heating the stillage to a temperature of 190° F. to 300° F., using all or a portion of the stillage as a component of a media, and using the media for a process chosen from the group consisting of fermentation and biomass production.

### BACKGROUND OF THE INVENTION

1. Technical Field

The present invention relates to methods of improving a fermentation process by addition of hydrothermally treated stillage as a media component to a fermentation process or upstream operation.

2. Background Art

The desire for alternative liquid motor fuel supplies has risen considerably over the past several decades. This has been driven by concern over climate change, crude oil supply stability, and energy security. Ethanol has proven to be a viable substitution for petroleum derived gasoline. It reduces harmful air pollutants, lowers dependence on fossil fuels, and lowers carbon emissions. The total world ethanol production in 2012 was 21.81B gallons, with the majority being produced in The United States and Brazil, 13.3B gallons and 5.58B gallons, respectively (US Department of Energy, Office of Energy Efficiency and Renewable Energy).

In the United States, corn is the most common feedstock, although wheat, milo, sweet sorghum, sugar beets and sugar cane are also used.

Ethanol fermentation is the biological process by which sugars are metabolized by yeast into ethanol and carbon dioxide. When grains are used as a feedstock, the starch must first be converted to sugars. The grain is milled to a flour, slurried to create a mash, and treated with enzymes to convert the starch to sugars. Yeast is added to the mash to ferment the sugars into beer in large fermenters. The ethanol is stripped from the beer and distilled. The residual spent grains, referred to as whole stillage, contain corn germ, corn bran, corn oil, unconverted starch, unfermented sugars, yeast cells, yeast metabolites and other suspended and dissolved solids. The whole stillage is generally separated into thin stillage and wet cake.

When sweet sorghum, sugar beets and sugar cane are used as a feedstock, sugar is extracted from the feedstock and fermented and distilled as described above. The residual product from the ethanol stripping process derived from sugar feedstocks is sometimes referred to as vinasse. For the sake of convenience, the residue of the ethanol stripping process, whether from a starch source or sugar source, and whether or not some or all of the solids are removed is hereafter referred to as “stillage”.

Stillage has been investigated for enhancing biological processes. For example, in the prior art ethanol process, stillage is recycled to the front end as make-up water in the slurry. The recycled stillage is referred to as “backset”. The proteins and nutrients in the stillage have been recognized as aiding fermentation, however this benefit is marginal. Therefore, there is a need for treating stillage to increase its value in a biological process.

A number of biological and non-biological methods have been developed for the improvement of thin stillage. Jacob P. Tewalt, et al. in WO2012/122393, assigned to POET Research Inc., disclose a method to clarify thin or whole stillage by growing fungi. Wicking, et al. in U.S. Patent Application Publication No. 2012/2094981 assigned to North American Protein Inc. disclose the use of fungi to remove inhibitory compounds from stillage and create a treated backset having improved ethanol fermentation performance.

M. Reaney, et al. in U.S. Patent Application Publication No. 2011/0130586, assigned to the University of Saskatchewan, disclose a method of recovering a recyclable water from thin stillage or dewatered (concentrated) thin stillage by polar solvent and/or oil extraction of microbial inhibiting metabolites such as glycerol, lactic acid and 2-phenylethanol (PEA) and the phospholipid α-glycerylphosphorylcholine (GPC) which has potential value in pharmaceutical applications.

J. Jump, et al. in U.S. Pat. No. 7,641,928, assigned to Novozymes North America Inc., disclose the use of enzymes to treat stillage and improve the dewatering properties of stillage.

J. Van Leeuwen, et al. in U.S. Patent Application Publication No. 2010/0196994, assigned to Iowa State University, disclose a method of continuous fungi cultivation on thin stillage to produce useful products and remediated water with significantly reduced COD (chemical oxygen demand).

Prior art processes have tried to remove suspended solids from thin stillage with various flocculating, coagulating, or precipitating additives and chemical agents. J. Hughes, et al., in U.S. Pat. No. 8,067,193, assigned to Ciba Specialty Chemicals, disclose the use of anionic polymer additives to increase coagulation and precipitation. D. W. Scheimann and A. S. Kowalski in U.S. Patent Application Publication No. 2006/0006116, assigned to Nalco Company, disclose methods of coagulating and flocculating thin stillage suspended solids using anionic polymer flocculants, cationic coagulants, and microparticulate settling aids and removing said suspended solids from the thin stillage. J. Collins, et al. in U.S. Patent Application Publication No. 2012/125859, also assigned to Nalco Co., disclose a method involving ionic flocculants for conditioning and processing whole or thin stillage to aid in the separation and recovery of protein and oil fractions. C. Griffiths in U.S. Patent Application Publication No. 2007/0036881, assigned to Ciba Specialty Chemicals, discloses the removal of suspended solids from thin stillage by treatment with polyacrylamide and electrocoagulation. Verkade, et.al. in U.S. Patent Application Publication No. 2009/0110772, assigned to Iowa State University, describe separating solids from a processed broth through chemical reaction with a phosphorous oxoacid to increase the water solubility of insoluble cellulosic, melaninic, ligninic, or chitinic solids.

Various filtration, microfiltration, and ultrafiltration processes have been disclosed in the prior art. Bento, et al. in U.S. Pat. No. 5,250,182, assigned to Zenon Environmental Inc., disclose a step-wise membrane separation process to recover lactic acid and glycerol together, from thin stillage in an ethanol stream. The stepwise process consists of ultrafiltration (UF), nanofiltration (NF) and reverse osmosis membrane units. Demineralized water can be recycled to fermentation or to boiler water make-up feed. Bento et al. suggest that the use of the membrane separation process in the production of ethanol based on the dry-milling of corn, substantially reduces or eliminates the use of a conventional evaporator.

A number of methods have been developed involving heat-treating stillage or a combination of heat-treating and chemical addition to stillage for the purposes of separation of solids and recovery of fermentation by-products, such as distiller's grains and syrup and especially oil. C. Brown in U.S. Pat. Nos. 2,216,904 and 2,216,905, assigned to The Sharples Corp., discloses the pre-treatment of “fermentation slop” by pressurized heat treatment to enhance agglomeration of slop solids, subsequently separating solids by filtration, settling or centrifugation and thereafter recovering oil from the solids by mechanical pressing or solvent extraction. In related U.S. Pat. No. 2,263,608, C. Brown further describes heat-treating of fermentation slops followed by separation and alkaline treatment of the settled solids to precipitate additional solids, filtering the precipitated solids.

Similarly, Woods et al. in U.S. Patent Application Publication No. 2011/0275845, assigned to Primafuel, disclose preparation of a clarified aqueous phase through the use of filters after separation of hot (140-212° F.) concentrated thin stillage into a light oil phase and a heavy aqueous phase and treating the oil phase with alkali chemicals including spent clean in place (CIP) solutions

U.S. Patent Application Publication No. 2009/0250412 and U.S. Pat. No. 7,608,729 to Winsness, et al., disclose methods for recovering oil from stillage concentrate including oil resulting from a process used for producing ethanol from corn. In one embodiment, the method includes heating the stillage concentrate to a temperature sufficient to at least partially separate, or unbind, the oil therefrom. The heating step includes heating to a temperature above 212 degrees F. but less than about 250 degrees F. The process disclosed by Winsness, et al. does not include treatment of un-concentrated stillage streams, nor does it disclose application of the thermal treatment method for improved fermentation.

U.S. Pat. No. 6,106,673 to Walker discloses a process and system for the separation of a fermentation process byproduct into its constituent components and for the subsequent recovery of those constituent components. The claimed process requires 1) mixing a starting mixture containing ethanol byproducts with a liquid (water) to form a diluted mixture, 2) heating of the diluted mixture containing the byproducts so as to separate the oil from a base component (fiber) of the byproduct to which the oil is bound at a temperature from about 140 degrees F. to about 250 degrees F., followed by 3) recovering oil, the base product (fiber), and possibly other substances such as molasses from the mixture. No claim to recycle of recovered water to fermentation or improvement of fermentation rate or yield by recycle of any or all of the liquor stream to upstream operations is made in Walker '673.

Other efforts have involved heat treatment and filtration of depleted lignocelluosic fermentation hydrolysate broth to separate undissolved solids from the liquid phase and create a low solids liquid (Hennessey, et.al., U.S. Patent Application Publication No. 2012/0178976 and Hennessey, et.al., U.S. Patent Application Publication No. 2012/0102823, assigned to Dupont).

While heating of stillage with or without chemical addition and mechanical separation described in prior art provides some separation of stillage co-products, it was not recognized that the use of all or a portion of hydrothermally treated stillage as a media component can improve fermentation processes.

Thermal hydrolysis has been investigated as a pretreatment step prior to anaerobic digestion of biomass, in particular the anaerobic digestion of waste activated sludge from biological waste water treatment facilities and the pretreatment of cellulosic biomass prior to enzymatic hydrolysis to liberate cellulosic sugars. The former has been commercially implemented while the latter remains a research and development endeavor. Camacho, et al. (Proceedings of the WEFTEC® 2008 Conference, Chicago, Ill. Water Environment Federation) reviewed the use of thermal hydrolysis as a pretreatment to anaerobic digestion of activated sludge and noted the improvements in both sludge dewaterability and biogas yield during anaerobic digestion. Optimal treatment temperatures were generally in the range of 150-200° C. (302-392° F.).

It is recognized that the temperatures utilized for hydrothermal pretreatment of municipal waste prior to anaerobic digestion are generally greater (300-450° F.) than those proposed for treating stillage in the present invention (190-300° F.).

Various methods have been proposed for utilizing stillage for biological purposes other than ethanol fermentation. M. Kriesler and D. Winsness in U.S. Patent Application Publication No. 2010/0028484, assigned to GS Cleantech, disclose methods for producing lipids from various stillage streams by the yeast Rhodotorula glutinis. Kriesler and Winsness also disclose conditioning of the stillage feedstocks by various pre-treatment methods including steam explosion, autohydrolysis, ammonia fiber explosion, acid hydrolysis, sonication and combinations thereof prior to inoculation with the lipid producing microorganism.

M. Ringpfeil in U.S. Pat. No. 5,981,233, assigned to Roche Vitamins Inc., discloses a process for manufacturing a xylanase enzyme complex from pre-treated thin stillage of rye, where the pretreatment includes removing solids from the rye thin stillage, evaporation of water, adding other nutrient components, and autoclaving prior to culturing the enzyme producing organism (Trichoderma).

In summary of the prior art, methods for improving fermentation by addition of stillage which has been hydrothermally treated in the range of 190-300° F. has not been described or suggested in patents or literature. It has been discovered for the first time herein that hydrothermally treating stillage and adding the treated stillage to a fermentation process increases fermentation rates and titers. Therefore, it is shown herein that the present invention provides a simple method for improving fermentation by the addition of hydrothermally treated stillage.

### SUMMARY OF THE INVENTION

The present invention provides for a method of improving fermentation by increasing the bioavailability of components in stillage, using all or a portion of the stillage as a component of a media, and using the media for a process such as fermentation or biomass production.

The present invention also provides for the metabolites and biomass recovered from the method above.

The present invention further provides for media including hydrothermally treating stillage obtained by heating the stillage to a temperature of 190° F. to 300° F.

The present invention also provides for a method of improving fermentation by removing solids in stillage, hydrothermally treating the stillage by heating the stillage to a temperature of 190° F. to 300° F., using all or a portion of the stillage as a component of a media, and using the media for a process chosen from the group consisting of fermentation and biomass production.

### DESCRIPTION OF THE DRAWINGS

Other advantages of the present invention are readily appreciated as the same becomes better understood by reference to the following detailed description when considered in connection with the accompanying drawings wherein:

FIG. 1 is a flowchart of a prior art ethanol fermentation process;

FIG. 2 is a flowchart of the hydrothermal treatment process of the present invention added after distillation, removing solids from the treated whole stillage and recycling the treated whole stillage to the front end of the ethanol process;

FIG. 3 is a flowchart of the hydrothermal treatment of the present invention added after separating whole stillage into thin stillage and wet cake and recycling the hydrothermally treated thin stillage to the front end of the ethanol process;

FIG. 4 is a flowchart of the hydrothermal treatment process of the present invention including removal of suspended solids from the hydrothermally treated thin stillage, creating stickwater and recycling the treated stickwater to the front end of the ethanol process;

FIG. 5 is a flowchart of the hydrothermal treatment process of the present invention including removal of suspended solids from heat treated thin stillage, creating stickwater and adding the stickwater to a microorganism fermentation process;

FIG. 6 is a flowchart of the hydrothermal treatment process of the present invention including a biological and/or chemical treatment for the removal of deleterious solids from the stickwater;

FIG. 7 is a flowchart of the hydrothermal treatment process of the present invention applied to thick stillage, wherein thick stillage is prepared by subjecting whole stillage to a particle size reduction process followed by separation of the whole stillage into wet cake and thick stillage, and following hydrothermal treatment, additional solids are removed from the reduced particle size, hydrothermally treated thick stillage to produce stickwater and the treated stickwater is recycled to the front end of the ethanol process; and

FIG. 8 is a flowchart of the hydrothermal treatment process of the present invention applied to thick stillage, wherein thick stillage is prepared by removing a first cut of solids from whole stillage separating the whole stillage into thin stillage and first cut solids, subjecting the first cut solids to a particle size reduction step, combining the size-reduced first cut solids and thin stillage, performing a second separation step on the combined first cut solids and thin stillage to form thick stillage and wet cake, and following hydrothermal treatment, additional solids are removed from the hydrothermally treated thick stillage to produce stickwater and the treated stickwater is recycled to the front end of the ethanol process.

### DETAILED DESCRIPTION OF THE INVENTION

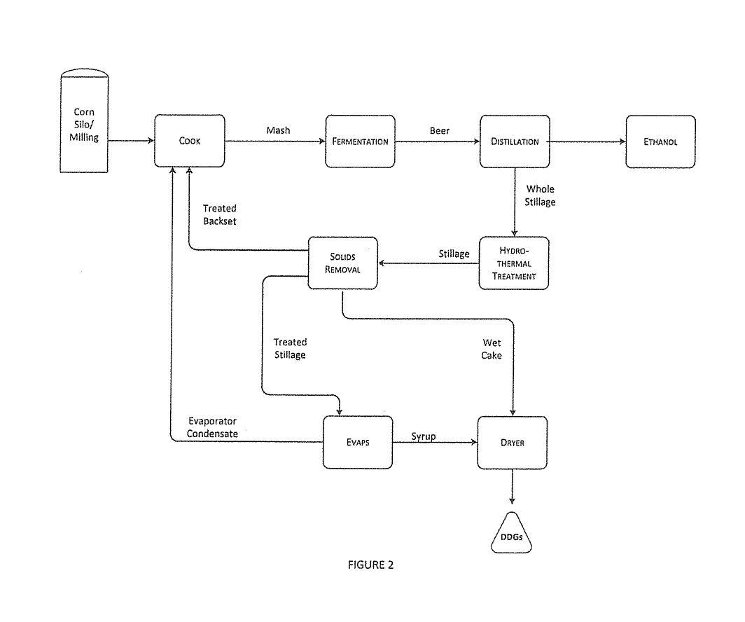

Most generally, the present invention provides for methods of fermentation that include the hydrothermal treatment of stillage and adding all or a portion of the treated stillage to enhance a fermentation media and to improve the overall fermentation process. These methods are shown in FIGS. 2-6. It should also be understood that while the FIGURES depict ethanol fermentation, the present invention is not limited to ethanol fermentation.

The term “hydrothermal treatment”, as used herein refers to heating a liquid stream, i.e. stillage, to a temperature of 190° F. to 300° F.

The term “stillage” as used herein, refers to a cloudy liquid produced during ethanol fermentation that includes solids that are not fermentable, solubles, oils, organic acids, salts, proteins, and various other components.

The term “whole stillage” is the effluent of water, suspended solids, and dissolved solids that exits the ethanol stripping process and has not been subjected to a downstream separation process. Whole stillage typically contains 10%-13% total solids w/w.

The term “thick stillage” refers to stillage having a solids composition intermediate to that of whole stillage and thin stillage. Thick stillage can be produced by a various methods including but not limited to removing and/or classifying the suspended solids from whole stillage, concentrating thin stillage, and addition of solids to thin stillage. Methods of producing thick stillage are described in more detail later in this section. Thick stillage typically contains 7%-11% total solids w/w.

The term “thin stillage” refers to a portion of the whole stillage where some or all of the suspended solids have been removed. Thin stillage can be produced as in the prior art by subjecting whole stillage to decanting centrifugation or by some other method to remove some or all of the solids such as, but not limited to filtration, dissolved air flotation, membrane separation, or hydrocyclone separation. Thin stillage typically contains 4%-8% total solids w/w.

The term “fermentation effluent” refers to the effluent stream of a fermentation step used to produce a metabolite or biomass. The effluent typically contains residual nutrients and metabolic by-products whether the product metabolite or biomass have been removed or not.

The term “backset” refers to that portion of stillage that is returned to fermentation or an upstream operation to improve water reuse and utilize residual nutrients in the stillage. The balance of thin stillage is typically concentrated in evaporators to produce syrup that is mixed with wet distiller's grains (WDG) and dried to produce distiller's dry grain with solubles (DDGS), a valuable co-product of the dry grind ethanol plant.

The term “stickwater” as used herein, refers to a fraction of the hydrothermally treated stillage stream obtained by removal of suspended solids. Stickwater is mainly water and soluble and typically contains less than 1 wt % suspended solids or less than 50% of the suspended solids in the stillage from which it is derived.

The term “fermentation” as used herein, refers to a biological process, either anaerobic or aerobic, in which suspended or immobilized microorganisms or cultured cells in a suitable media are used to produce metabolites and/or new biomass.

Stillage is a complex mixture of yeast cells, proteins, fiber, lipids, minerals, salts, organic acids, glycerol, monosaccharides, and oligosaccharides. Many of the components of the stillage are useful to microorganisms, but are not bio-available. The process of hydrothermal treatment converts or releases these components to increase their bioavailability and enhance fermentation.

For example, stillage contains many oligosaccharides, polymers that cannot be metabolized by many microorganisms. The hydrothermal treatment of stillage hydrolyzes the oligosaccharides into monosaccharides and disaccharides.

The proteins in stillage are present in tight matrixes. These matrixes bind the proteins, phosphates, sugars, cations, anions, metals, salts and amino acids. The hydrothermal treatment of stillage unfolds the proteins in a way that increases the bioavailability of these components by unlocking the protein matrices including the phosphates, sugars, cations, anions, metals, salts, and amino acids. For example, betaine is known to enhance fermentations. Betaine is present in stillage but locked in these complex protein matrixes. The hydrothermal treatment of stillage releases the betaine such that it is available to the microorganisms. In general, the hydrothermal treatment of stillage denatures and hydrolyzes proteins, increasing their bioavailability. The denaturation of the protein can affect the quaternary structure (dissociating sub-units and/or spatial arrangements), tertiary structure (disrupting covalent, noncovalent, or Van der Waals interactions between amino acid side chains), or secondary structure (dissociating regular repeating patterns into random coils). Loss of function can also occur (such as with enzymes). Hydrolysis of proteins causes the break down into component amino acids.

Stillage also contains corn oil. Corn oil adheres to microorganism cells retarding their ability to convert carbon into biomass or metabolites. The hydrothermal treatment of stillage reduces the corn oil emulsion in stillage making the oil easier to extract. The extraction of oil from stillage reduces the negative effect corn oil has on fermentation.

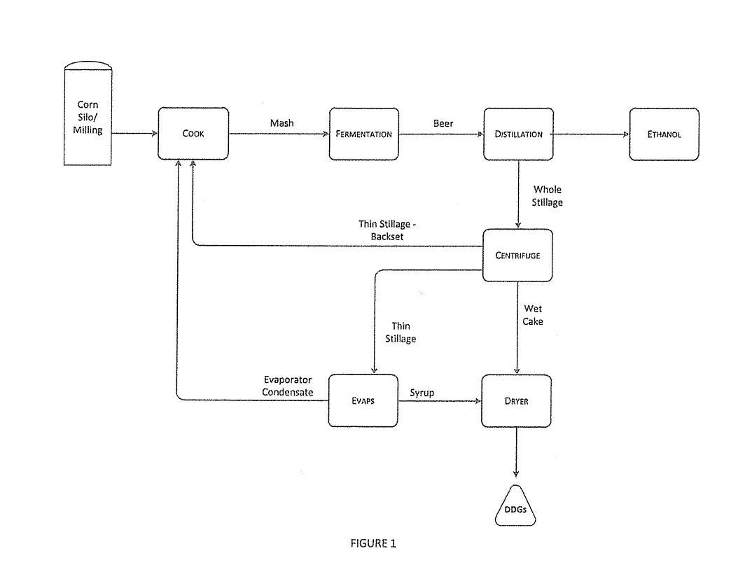

In the method of ethanol fermentation, the corn is milled, slurried with water comprised of fresh water, recycled process condensate, process water and recycled thin stillage (backset) and cooked to obtain a mash, fermented to obtain a beer, distilled to produce ethanol and obtain stillage as a bottoms product of the beer column as shown in FIG. 1, labeled “Prior Art”. Then, once stillage has been produced, the stillage processing method of the present invention can be introduced at different points as further detailed below.

FIG. 2 shows one embodiment of the present invention where the hydrothermal treatment method is applied to whole stillage. First, whole stillage is heated by a heating mechanism, such as, but not limited to, a heat exchanger or steam injection, to a temperature of 190° F. to 300° F. and held in a reactor for 3 to 180 minutes. The reactor pressure is maintained at or above the saturation pressure of the stillage. Afterwards, preferably, the stillage is cooled below its atmospheric boiling point, and preferably below 212 degrees F. Preferably, solids are removed and the hydrothermally treated stillage is recycled to fermentation or an upstream step.

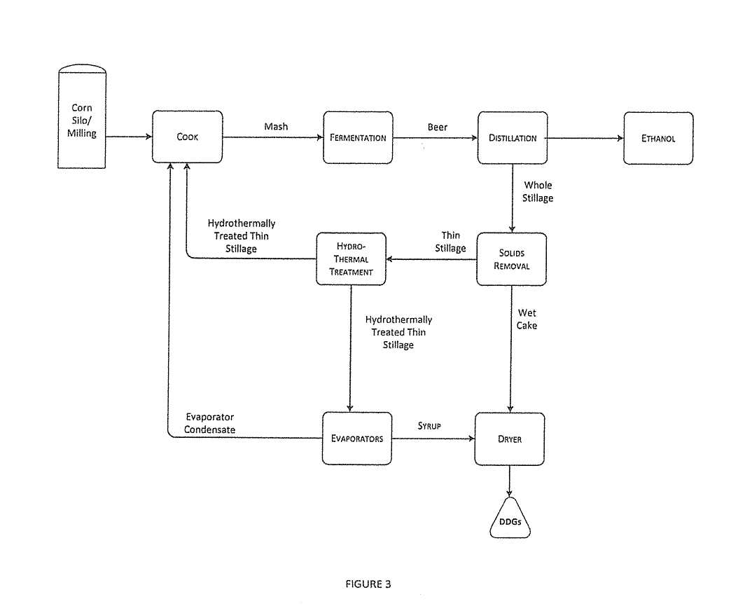

In another embodiment of the present invention, the hydrothermal treatment process is applied to stillage having variable solids concentration. Solids can be removed from stillage by one or more processes such as, but not limited to centrifugation, decanting centrifugation, filtering centrifugation, dissolved air flotation, filtration, membrane separation, quiescent decantation, and hydroclonic separation. Various chemical and solid agents can be added and the temperature can be adjusted to enhance removal of solids. The stillage can also be diluted before or after the hydrothermal treatment step. As shown in FIG. 3, the process of the present invention can be applied to thin stillage. Thin stillage is heated by a heating mechanism, such as, but not limited to, a heat exchanger or steam injection, to a temperature of 190° F. to 300° F. and held in a reactor for 3 to 180 minutes. The reactor pressure is maintained at or above the saturation pressure of the stillage. Afterwards, preferably, the stillage is cooled below its atmospheric boiling point, and preferably below 212 degrees F. The hydrothermally treated thin stillage is recycled to fermentation or an upstream step.

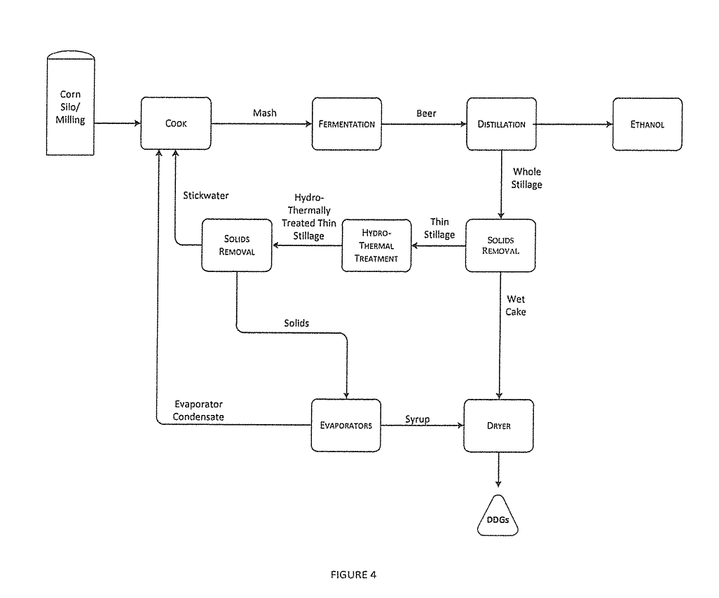

In another embodiment of the present invention, the hydrothermally treated thin stillage can be further clarified by removal of suspended solids as shown in FIG. 4 to create a “stickwater” fraction. All or a portion of the hydrothermally treated and clarified stickwater can be recycled to fermentation or an upstream step.

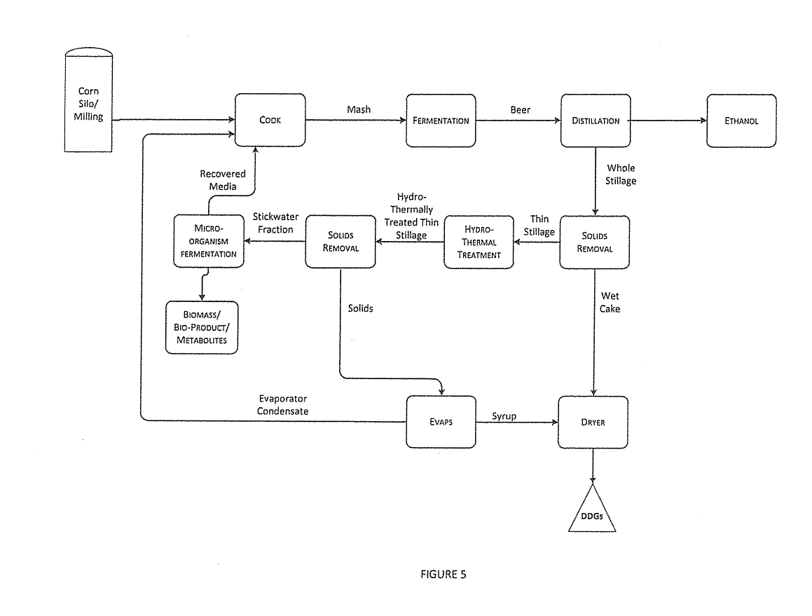

Another embodiment of the present invention is shown in the flowchart of FIG. 5. The hydrothermally treated stickwater is added to a microorganism fermentation or cell culture process. The effluent from the microorganism fermentation or cell culture process can be added to additional fermentation processes including recycling to the cook step of an ethanol process.

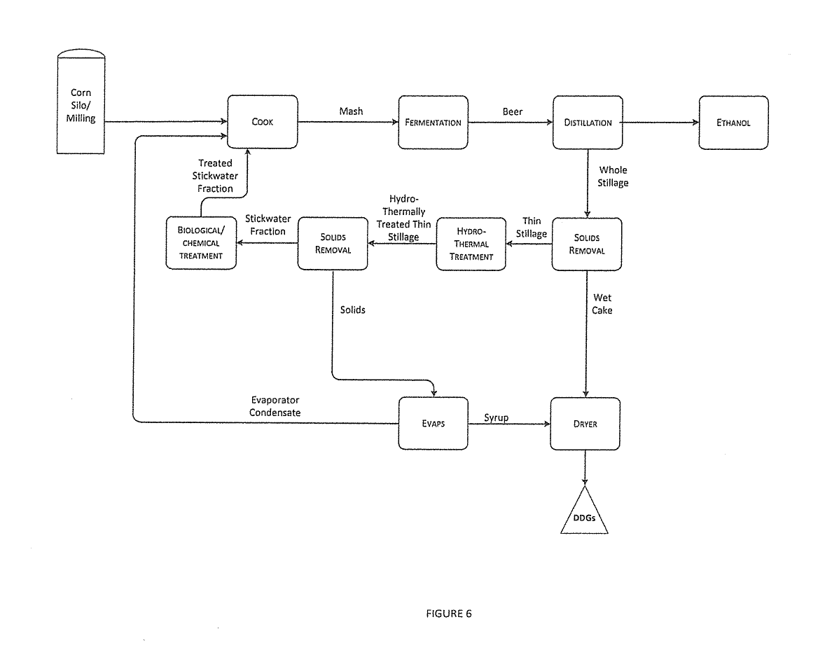

Another embodiment of the present invention is shown in the flowchart of FIG. 6. The hydrothermally treated stickwater is further treated to remove fermentation inhibitors. Fermentation inhibitors are removed by one or more of the following processes; biological digestion or other biological treatment, filtration, membrane separation, precipitation, solvent extraction, ion exchange, distillation, electro-dialysis, or other chemical treatment.

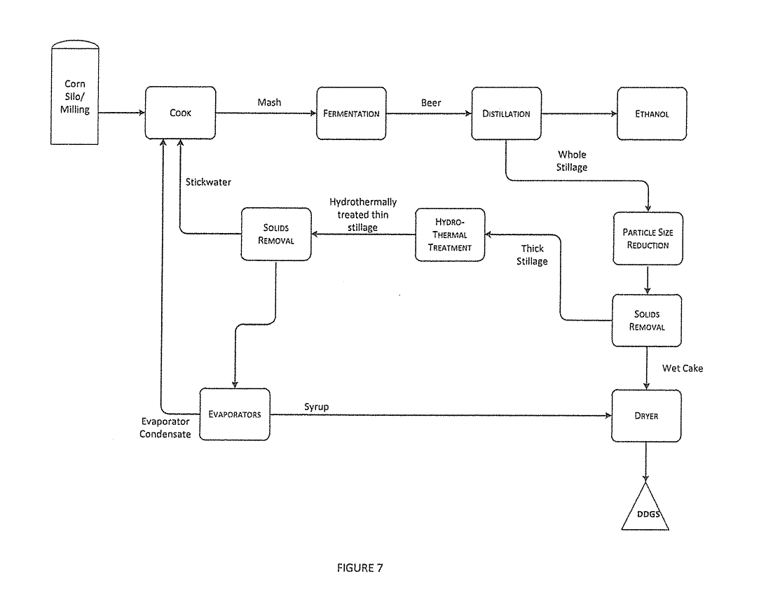

Another embodiment of the present invention in which thick stillage is hydrothermally treated is shown in the flowchart of FIG. 7. In this embodiment, thick stillage is first produced by subjecting all or a portion of the whole stillage to a particle size reduction process prior to whole stillage centrifugation. The particle size reduction process produces fine suspended solids that reduces solids in the centrifuge wet cake and increases solids in the centrate, thus producing thick stillage. The presence of fine solids in the thick stillage allows for improved rates of reactions and release of fermentation enhancers in the hydrothermal treatment step as well as increased oil recovery downstream. Methods of particle size reduction can be chosen from many methods known to those skilled in the art, including, but not limited to, colloid mills (e.g. ball mills, bead mills), disc mills, pin mills, jet mills, rotor-stator mixers, high-pressure homogenizers, steam explosion, and ultra-sonication. Optionally, the fine solids can be removed from the hydrothermally treated thick stillage prior to adding all or a portion of the hydrothermally treated thick stillage to the fermentation step or an operation upstream of fermentation. Methods for separating the fine solids from the hydrothermally treated thick stillage include, but are not limited to, centrifuges, decanting centrifuges, filter centrifuge, filters, membranes, hydrocyclone, quiescent decantation, dissolved air floatation, and flocculation.

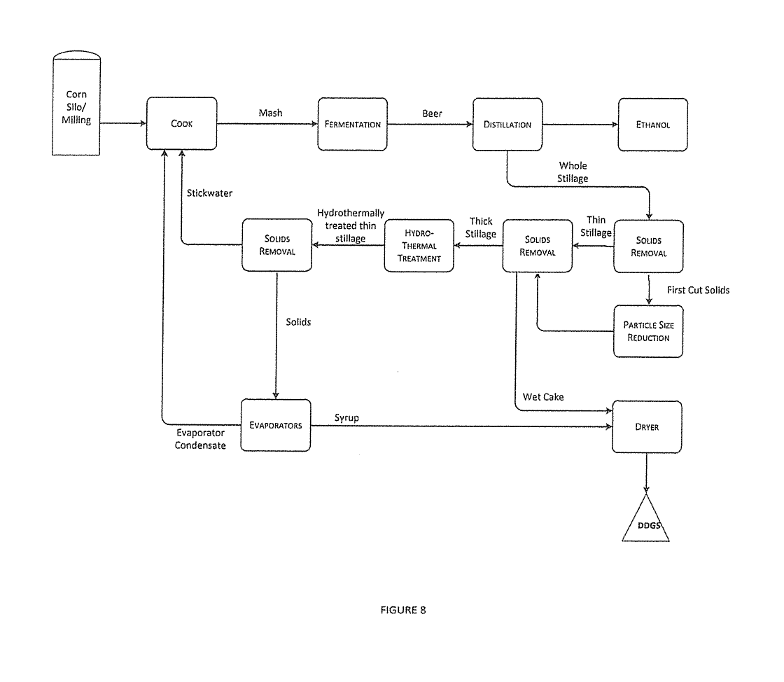

Another embodiment is shown in the flowchart of FIG. 8. In this embodiment, the hydrothermal treatment process of the present invention applied to thick stillage, wherein thick stillage is prepared by removing a first cut of solids from whole stillage thus separating the whole stillage into thin stillage and first cut solids, subjecting the first cut solids to a particle size reduction step, combining the size-reduced first cut solids and thin stillage, followed by a second separation step performed on the combined first cut solids and thin stillage to form thick stillage and wet cake. Following hydrothermal treatment of the thick stillage, additional solids are removed from the hydrothermally treated thick stillage to produce stickwater and the treated stickwater is recycled to the front end of the ethanol process.

There are two advantages to the processes of the present invention described herein over prior art processes. First, there are components in the stillage that are fermentation enhancers, such as the phosphates, sugars, cations, anions, metals, salts, and amino acids trapped in the proteins as described above. Insoluble components in the stillage can be fermentation enhancers when solubilized by the present invention.

Second, the solids in the stillage are typically not fermentable and reduce the amount of new carbon source that can be added to the media. By reducing suspended solids by the methods described below and increasing fermentation enhancers (i.e. increasing the bioavailability of stillage components) with the hydrothermally treated stillage or stickwater of the present invention, fermentation rates and final titers can be increased.

Therefore, in summary, the present invention provides for a method of improving ethanol fermentation by adding hydrothermally treated stillage to fermentation media in a fermentation step or adding in an operation upstream of fermentation.

More specifically, the method of improving fermentation includes the steps of increasing the bioavailability of components in stillage; preferably by hydrothermally treating stillage by heating the stillage to a temperature of 190° F. to 300° F., using all or a portion of the hydrothermally treated stillage as a component of a media, and using the media for a process such as fermentation or biomass production.

The step of increasing the bioavailability and the step of hydrothermally treating the stillage are described above. The stillage can be whole stillage, concentrated stillage, diluted stillage, or any other type of stillage as described above. The stillage can be diluted with a composition such as, but not limited to, water, process water, steam, or process vapor. The process vapor can include, but is not limited to, flash steam, distillation vapor, distillation vapor condensate, evaporated thin stillage vapor, evaporated thin stillage vapor condensate, evaporated stickwater vapor, evaporated stickwater vapor condensate, dryer vapor, or dryer vapor condensate.

The fermentation improved is preferably an alcohol fermentation. The fermentation can produce a metabolite such as, but not limited to, organic acids, alcohols, lipids, carbohydrates, proteins, and secondary metabolites. The fermentation can be anaerobic or aerobic. The biomass can be, but is not limited to, algae, bacteria, yeast, fungi, archae, other microorganism, or cultured cells.

The hydrothermally treated stillage can be produced from stillage of various solids concentrations including whole and thin stillage as described above. The solids removed can include, but are not limited to, suspended solids, dissolved solids, oil, proteins, fiber, and ash. Suspended solids can be further removed from hydrothermally treated stillage by a method including but not limited to, centrifuges, decanting centrifuges, filter centrifuge, filters, membranes, hydrocyclone, quiescent decantation, dissolved air floatation, or flocculation to produce a stickwater fraction. Dissolved solids can further be removed by membranes, biological remediation, electro-dialysis, ion exchange, distillation, solvent extraction, or precipitation. All or a portion of the treated stillage or stickwater can be recycled to fermentation or an operation upstream of fermentation.

Organic compounds in the hydrothermally treated stillage can provide all or a portion of the carbon source in the media and can provide all or a portion of the nutrient requirements. Also, the fermentation media can be supplemented by adding additional carbon and nutrient sources. The carbon source can be, but is not limited to, dextrose, sucrose, fructose, xylose, arabinose, other carbohydrates, organic acids, glycerol, ethanol, other alcohols, carbon monoxide, carbon dioxide, methane, or other hydrocarbons. The carbon source can be derived from cellulosic material.

The hydrothermally treated stillage can make up all or a portion of the media.

One or more agents can be added to the stillage to assist in the removal of solids, such as, but not limited to, acids, bases, minerals, polymeric flocculants, microparticulate settling aids (diatomaceous earth, bentonite, montmorillonite, colloidal silica borosilicate, and microsand), precipitation aids, and salts. The temperature can also be adjusted to assist in the removal of solids.

Whether the stillage is thin stillage or whole stillage, at least part of the solids can be removed from the thin stillage or whole stillage before or after the hydrothermal treatment step.

Therefore, the present invention provides for a method of improving fermentation by removing solids in stillage, hydrothermally treating the stillage by heating the stillage to a temperature of 190° F. to 300° F., using all or a portion of the stillage as a component of a media, and using the media for a process chosen from the group consisting of fermentation and biomass production.

The stillage can also be thick stillage produced by a method such as, but not limited to, removal of water from thin stillage to concentrate solids, filtration of thin stillage to concentrate solids, centrifugation of whole stillage under centrifuge operating conditions that promote transport of more solids into the centrate and less into the wet cake fraction, addition of solids to thin stillage, particle size reduction of stillage to allow for additional fine solids to be transported into the centrate fraction during centrifugal separation of stillage, and combinations thereof. Some or all of the solids can be removed from the thick stillage prior to or after hydrothermal treatment.

The method can further include reducing the particle size of all or a portion of the stillage prior to or after hydrothermal treatment. The particle size reduction can be performed on thin stillage, whole stillage, wet cake, or thick stillage. The particle size reduction can be accomplished by a process such as, but not limited to, colloid mills, disc mills, pin mills, jet mills, rotor-stator mixers, high-pressure homogenizers, or ultra-sonication. This method can further include the step of removing some of the solids from the stillage prior to or after said particle size reduction step. The removed solids can be added back to the stillage after the particle size reduction. The solids can also be removed from the stillage after the hydrothermally treating step. Any removal of solids can be accomplished by centrifuges, decanting centrifuges, filter centrifuge, filters, membranes, hydrocyclone, quiescent decantation, dissolved air floatation, or flocculation.

The method can also include removing at least part of the oil from the stillage before or after the hydrothermal treatment step.

Metabolites can be separated from the fermentation media, and biomass can be recovered from the media.

The fermentation effluent can also be used in an additional fermentation process, such as in alcohol fermentation. The biomass and/or metabolites can be recovered prior to the additional fermentation process.

The present invention also provides for the metabolites and biomass recovered from the method above, including, but not limited to the biomass of bacteria, yeast, algae, archae, fungi, and cultured cells and metabolites such as, but not limited to alcohols, organic acids, lipids, carbohydrates, proteins and secondary metabolites.

The present invention further provides for media including hydrothermally treating stillage obtained by heating the stillage to a temperature of 190° F. to 300° F.

The present invention also provides for a method of performing ethanol fermentation by separating whole stillage into wet cake and stillage, hydrothermally treating the stillage by heating the stillage to a temperature of 190° F. to 300° F., and adding all or a portion of the treated stillage to the ethanol fermentation step or an operation upstream of fermentation.

The method can further include the steps of producing stickwater by removing additional suspended solids from the hydrothermally treated stillage by a method such as, but not limited to, centrifuges, decanting centrifuges, filter centrifuge, filters, membranes, hydrocyclone, quiescent decantation, dissolved air floatation, or flocculation; and adding all or a portion of the stickwater to an ethanol fermentation step or an operation upstream of fermentation.

The present invention further includes a method of performing ethanol fermentation by separating whole stillage into a first cut solids stream and thin stillage stream, reducing the particle size on all or a portion of the first cut solids, returning the reduced particle size solids to the thin stillage stream to produce thick stillage, hydrothermally treating the thick stillage by heating to a temperature of 190° F. to 300° F., and adding all or a portion of the treated stillage to the ethanol fermentation step or an operation upstream of fermentation.

The present invention also provides for a method of increasing bioavailability of stillage components to microorganisms by hydrothermally treating stillage by heating the stillage to a temperature of 190° F. to 300° F., increasing the bioavailability of components in the stillage, adding the hydrothermally treated stillage to media and providing to microorganisms. Increasing the bioavailability of components has been described above and is accomplished by hydrolyzing oligosaccharides into monosaccharides and disaccharides, unfolding protein matrixes, denaturing protein, hydrolyzing protein, and combinations thereof.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Ethanol Fermentation Improved by Stickwater Produced at 250° F. and 285° F.

In this example, both thin stillage obtained from a commercial ethanol plant and stickwater prepared thereof were used as the basis for a fermentation media to which anhydrous glucose was added as a carbon source. No other nutrients were added, thereby showing that the stickwater prepared by hydrothermal treatment at 250° F. or 285° F. can be a superior media component compared to thin stillage.

Procedures

Hydrothermal Treatment

Thin stillage obtained from a commercial ethanol plant was continuously pumped through a series of Plate and Frame Heat Exchangers (PHEs) into a stirred reactor. The PHEs heated the stillage to the target temperature of 250° F. or 285° F. The reactor's pressure was maintained at the saturation pressure of the stillage. The reactor had a mean residence time of 90 minutes and 40 minutes for the 250° F. and 285° F. runs respectively. The treated stillage was continuously withdrawn from the reactor and cooled to 185° F., then held in a quiescent decantation tank with a mean residence time of 40 minutes. The stickwater fraction was continuously removed from the decantation tank and collected. The remaining thin stillage was discarded. The volume ratio of Stickwater to the discarded fraction was 1:1.

Culture and Fermentation

The batch fermentations were started with an initial culture of a commercial ethanol producing yeast, Saccharomyces cerevisiae (Ethanol Red®, obtained from Fermentis division of Lesaffre). Two batches of stickwater were produced from commercial thin stillage based on the methods described above, and the resultant stickwater from each batch were then compared to an original sample of thin stillage for ethanol fermentation performance. To a 1 liter sample of thin stillage or stickwater, approximately 200 grams of anhydrous glucose was added as the carbon source and allowed to dissolve. The resultant glucose/sample was added to an autoclaved 1.5 liter stirred reactor (Pyrex® Pro-Culture Spinner Flask (1.5 L); Corning, Corning, N.Y.) and the temperature of the fermentor was equilibrated to 82° F. prior to inoculation.

Inoculum

The inoculum was prepared in a 250 ml sterile Erlenmeyer flask by addition of 1 gram of lyophilized yeast into 100 ml of filter sterilized 2% (w/w) malt extract broth and was incubated at 82° F. for 30 minutes before use. From the inoculum, 5 ml was used to start the fermentations.

Batch Fermentation

An initial sample was taken prior to inoculation and frozen. The fermentation was done at 82° F. with 110 rpm agitation. Fermentation vent locks were fitted to the fermenters at 1 hour after inoculation, to prevent oxygen from entering the vessel. At various time points, samples were removed and frozen prior to analysis via HPLC. The stickwater fermentations were stopped after 43.7 hours and the thin stillage fermentation was stopped at 48 hours.

Methods of Analysis

HPLC analysis for sugars and organic acids is based on NREL method LAP 015. Analysis was performed on a Phenomenex Rezex ROA-Organic acid column at 55° C. using 0.005 N sulfuric acid as the eluent and flow rate set at 0.6 ml/min. The detection was via a UV/Vis detector set at 190 nm and CAD (Charged Aerosol Detector). Samples were unthawed, diluted, filtered through a 0.2 micron nylon filter. The injection volume was 20 μl and the samples were compared against standards

Results and Discussion

TABLE 1 shows data obtained at the end of fermentation (48 hours) and demonstrates that stickwater produced by 250° F./90 minutes and 285° F./40 minutes hydrothermal treatment provides a superior fermentation medium for ethanol production as compared to thin stillage. In this set of fermentation runs, both 250° F./90 minutes and 285° F./40 minutes treatments yielded approximately 0.33 g ethanol/g dextrose whereas the thin stillage fermentation yielded 0.24 g/g, only 71% of the yield achieved in stickwater runs.

TABLE 1
Ethanol Fermentation with base media containing stickwater versus
untreated thin stillage

| | Base media prepared with . . . | | |
|---|---|---|---|
| | 250° F./90 min | 285° F./40 min | Untreated Thin |
| | Stickwater | Stickwater | Stillage |
| Ethanol weight yield | 0.326 | 0.333 | 0.235 |
| on dextrose | | | |
| (g ethanol/g dextrose) | | | |

### Example 2

### Ethanol Fermentation Enhanced with Hydrothermally Treated Thick Stillage

In this example, the flexibility of the present invention to produce advantageous stickwater from stillage of varying solids concentrations, i.e. thin stillage, thick stillage, and whole stillage, is demonstrated.

Procedures

Whole stillage and thin stillage were obtained from a commercial ethanol plant. To produce stillage having a suspended solids concentration between that of whole and thin, whole stillage was filtered through a series nylon filter bags of decreasing pore size (1000, 600, 400, 100 microns). Filtrate from the 100 micron filter was taken as “thick stillage”. Samples of thin stillage, whole stillage, and thick stillage were analyzed for total solids, suspended solids and oil. The resultant material was thermally conditioned at 270° F. for 40 minutes, and then separated by quiescent decantation to produce a stickwater fraction. The stickwater fraction was used as fermentation medium for ethanol production, as previously described in EXAMPLE 1.

Results and Discussion

The first data row of TABLE 2 gives suspended solids levels for each of the starting stillage streams prior to hydrothermal treatment and production of stickwater thereof. The remaining data rows of TABLE 2 show that media containing stickwater prepared by the present invention from any of the stillage concentrations can be used as fermentation media with no loss of performance. Although the ethanol yield differences observed in these runs is within typical variability for laboratory fermentations, there is an indication that stickwater produced from whole stillage can in fact offer enhanced performance. The ability to produce stickwater from thin, thick or whole stillage can provide the ethanol producer with advantageous fermentation yields and process flexibility.

TABLE 2
Suspended solids in whole, thick and thin stillage used to produce
stickwater and ethanol fermentation results using media produced
from the stickwater

| Total Suspended | Whole | Thick | Thin |
|---|---|---|---|
| Solids (w/w) | Stillage | Stillage | Stillage |
| | 8.64 | 3.56 | 1.83 |
| | Stickwater Source for Media | | |
| Fermentation | Whole | Thick | Thin |
| Results | Stillage | Stillage | Stillage |
| Dextrose Utilized | 181.5 | 173.1 | 190.7 |
| (g/l) | | | |
| Ethanol yield (g/g | 0.430 | 0.455 | 0.435 |
| dextrose utilized) | | | |
| Yield | 84.1% | 89.0% | 85.1% |

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method of improving fermentation, including the steps of:
increasing the bioavailability of components in stillage;
using all or a portion of the stillage as a component of a media; and
using the media for a process chosen from the group consisting of fermentation and biomass production.

2. The method of claim 1, wherein said increasing bioavailability step is further defined as a step chosen from the group consisting of hydrolyzing oligosaccharides into monosaccharides and disaccharides, unfolding protein matrixes, denaturing protein, hydrolyzing protein, and combinations thereof.

3. The method of claim 1, wherein said increasing the bioavailability further includes the step of hydrothermally treating the stillage by heating the stillage to a temperature of 190° F. to 300° F.

4. The method of claim 3, wherein said hydrothermally treating step is further defined as holding the stillage at the treatment temperature for 3 to 180 minutes and at a pressure at or above the saturation pressure of the stillage.

5. The method of claim 1, wherein the fermentation produces a metabolite chosen from the group consisting of organic acids, alcohols, lipids, carbohydrates, proteins, and secondary metabolites.

6. The method of claim 1, wherein the biomass is chosen from the group consisting of algae, bacteria, yeast, fungi, archae, and cultured cells.

7. The method of claim 3, wherein organic compounds in the hydrothermally treated stillage provide all or a portion of the carbon source in the media.

8. The method of claim 3, wherein the hydrothermally treated stillage provides all or a portion of the nutrient requirements.

9. The method of claim 1, further including the step of adding a composition to the media from the group consisting of a carbon source and nutrients.

10. The method of claim 9, wherein the carbon source is chosen from the group consisting of dextrose, sucrose, fructose, xylose, arabinose, other carbohydrates, organic acids, glycerol, ethanol, other alcohols, carbon monoxide, carbon dioxide, methane, and a carbon source derived from cellulosic material.

11. The method of claim 3, wherein the hydrothermally treated stillage makes up all or a portion of the media.

12. The method of claim 1, wherein the stillage is chosen from the group consisting of thin stillage, whole stillage, and thick stillage.

13. The method of claim 12, wherein the stillage is thick stillage and is produced by a method chosen from the group consisting of: removal of water from thin stillage to concentrate solids, filtration of thin stillage to concentrate solids, centrifugation of whole stillage under centrifuge operating conditions that promote transport of more solids into the centrate and less into the wet cake fraction, addition of solids to thin stillage, particle size reduction of stillage to allow for additional fine solids to be transported into the centrate fraction during separation of stillage, particle size reduction of the corn slurry prior to fermentation to allow for additional fine solids to be transported into the stillage fraction during separation, and combinations thereof.

14. The method of claim 1, further including the step of performing a particle size reduction process on all or a portion of the stillage prior to or after said hydrothermally treating step, wherein the stillage is chosen from the group consisting of thin stillage, whole stillage, wet cake, and thick stillage.

15. The method of claim 5, further including the steps of separating and recovering metabolites and recovering biomass from the fermentation media.

16. The method of claim 1, further including the step of using the fermentation effluent in an additional alcohol fermentation process.

17. The method of claim 3, further including the steps of producing stickwater by removing suspended solids from the hydrothermally treated stillage by a method chosen from the group consisting of centrifuges, decanting centrifuges, filter centrifuge, filters, membranes, hydrocyclone, quiescent decantation, dissolved air floatation, and flocculation; and adding all or a portion of the stickwater to an ethanol fermentation step or an operation upstream of fermentation.

18. The metabolites recovered from the method of claim 5.

19. The biomass recovered from the method of claim 6.

20. Media including hydrothermally treating stillage obtained by heating the stillage to a temperature of 190° F. to 300° F.

21. A method of improving fermentation, including the steps of:
removing solids in stillage;
hydrothermally treating the stillage by heating the stillage to a temperature of 190° F. to 300° F.;
using all or a portion of the stillage as a component of a media; and
using the media for a process chosen from the group consisting of fermentation and biomass production.

22. The method of claim 21, wherein said removing solids step is further defined as removing solids chosen from the group consisting of suspended solids, dissolved solids, oil, proteins, fiber, and ash.

23. The method of claim 21, wherein said removing step is performed by a mechanism chosen from the group consisting of centrifuges, decanting centrifuges, filter centrifuge, filters, membranes, hydrocyclone, quiescent decantation, dissolved air floatation, flocculation, biological remediation, electro-dialysis, ion exchange, distillation, solvent extraction, and precipitation.

24. The method of claim 21, further including the step of adding one or more agents to assist in the removal of solids chosen from the group consisting of acids, bases, minerals, polymeric flocculants, microparticulate settling aids, precipitation aids, and salts.

25. The method of claim 21, further including the step of adjusting the temperature to assist in the removal of solids.
